(19) United States
(12) Patent Application Publication
(10) Pub. No.: US 2006/0154255 A1
(43) Pub. Date: Jul. 13, 2006

(54) Blood and tissue sample assessment of mitochondrial function and biochemistry as a tool for selection for feed efficiency and other production parameters

(76) Inventors: Walter Bottje, Fayetteville, AR (US); Neil R. Pumford, Fayetteville, AR (US); Ron Okimoto, Fayetteville, AR (US); Mohammad Iqbal, Nottinham, MD (US)

Correspondence Address: ANGELA FOSTER, PHD, ESQ., 2906 BIRCHWOOD COURT, NORTH BRUNSWICK, NJ, 08902-3933, US

(21) Appl. No.: 10/745,874

(22) Filed: Dec. 27, 2003

Related U.S. Application Data
(63) Continuation-in-part of application No. PCT/US02/20048, filed on Jun. 26, 2002.

(51) Int. Cl. C12Q 1/68; G06F 19/00

(52) U.S. Cl. 435/6; 702/19

(57) ABSTRACT

The present invention provides methods for predicting feed efficiency in animals by correlating the association of mitochondrial function with feed efficiency. In one embodiment, the invention provides a method for determining feed efficiency comprising comparing proteins patterns and activity levels with antibody interaction of proteins associated with mitochondrial function and feed efficiency. In an alternative embodiment, the present invention also provides methods for predicting feed efficiency in animals by identifying animals having genetic mutations or polymorphisms that are associated with mitochondrial function and feed efficiency.

### 1. RELATED APPLICATIONS

This application claims the benefit of U.S. Provisional Application No. 60/301,157, filed on Jun. 27, 2001 and International Application No. PCT/US02/20448, filed on Jun. 27, 2002.

### 2. FIELD OF INVENTION

The present invention is related to feed efficiency. More specifically, this invention provides methods useful for predicting feed efficiency based upon the association of biochemical analyses of biological samples relating to mitochondrial function with feed efficiency.

### 3. BACKGROUND OF THE INVENTION

Feed efficiency remains one of the most important traits in commercial animal breeding programs. Moreover, feed represents approximately 60 to 70% of the cost of raising a broiler. To date, a biological explanation for differences in feed efficiencies between individual birds is lacking. Despite marked improvement in these traits, there remains significant within- and between-strain variations in growth and feed efficiency in broiler strains that include a 10% variation in broiler crosses for feed efficiency (Emmerson, 1997). For example, a 1991 broiler strain demonstrated a 250 to 300% improvement in body weight and feed efficiency (gain to feed) compared to a 1957 random bred control population (Havenstein et al., 1994). Variations in broiler growth performance and phenotypic expression of feed efficiency (Emmerson, 1997) may be due in part to differences or inefficiencies in mitochondrial function since the mitochondria is responsible for producing 90% of the energy for the cell as adenosine triphosphate (ATP),.

The respiratory chain/oxidative phosphorylation system on the inner mitochondrial membrane consists of four multi-protein complexes (I-IV) and ATP synthase (Complex V). Electron movement down the respiratory chain to the terminal electron acceptor, oxygen (O₂), is coupled to proton (H+) pumping from the matrix to the intermembrane space. The resulting proton-motive force drives ATP synthesis (from ADP and Pᵢ) as protons move back through the ATP synthase (Lehninger et al., 1993). Electrons enter the respiratory chain through NADH- or FADH-linked substrates such as glutamate and succinate at Complex I and II, respectively.

Mitochondrial function can be assessed by polarographic measurement of oxygen consumption (Estabrook, 1967). In the presence of energy substrate (e.g. glutamate or succinate), isolated mitochondria exhibits an initial slow rate of oxygen consumption designated State 2 respiration. Adenosine diphosphate (ADP) stimulates electron transport chain activity and initiates rapid oxygen consumption (State 3 respiration) that is followed by a slower rate of oxygen consumption (State 4 respiration) when ADP levels decline following oxidative phosphorylation to ATP. Functional indices calculated from these rates of oxygen consumption include the respiratory control ratio (RCR, an index of respiratory chain coupling) and ADP:O ratio (Estabrook, 1967). The RCR represents the degree of coupling or efficiency of electron transport chain activity and is calculated as State 3 divided by State 4 respiration rate. The ADP:O ratio is the amount of ADP per nanoatom of monomeric oxygen consumed during State 3 respiration, and is an index of oxidative phosphorylation. The acceptor control ratio (ACR), calculated as State 3 divided by State 2 respiration rate, can also be determined.

Mitochondrial inefficiency may occur as a result of leakage of electrons from the respiratory chain. For example, 2 to 4% of oxygen consumed by the mitochondria may be incompletely reduced to reactive oxygen species (ROS) such as superoxide (O₂^{o-}) and hydrogen peroxide (H₂O₂) due to univalent reduction of oxygen by electrons (Boveris and Chance, 1973; Chance et al., 1979). The mitochondrial formation of ROS makes this organelle a major source of oxidative stress in the cell. Therefore, if ROS are not metabolized by antioxidants, oxidation of critical structures in the mitochondria and/or cell such as lipids, proteins and DNA, can lead to further inefficiencies that accentuate additional ROS production.

Increased mitochondrial ROS production has been linked to various metabolic diseases (Fiegal and Shapiro, 1979; Hagen et al., 1997; Kristal et al., 1997; Herrero and Barja, 1998, Lass et al, 1998; Cawthon et al., 2001; Iqbal et al., 2001; Tang et al., 2001). The use of respiratory chain inhibitors can be employed to identify site-specific defects in electron transport within the mitochondria. Although electron leaks occur mainly within Complex I or III of the respiratory chain (Turrens and Boveris, 1980; Nohl et al., 1996; Herrero and Barja 1998), Kwong and Sohal (1998) demonstrated that sites of H₂O₂ production are tissue dependent. The findings of Kwong and Sohal (1998) may explain in part the findings of increased ROS production associated with Complex I and III in heart, muscle and lung mitochondria (Iqbal et al., 2001; Tang et al., 2001), and Complex II in liver mitochondria (Cawthon et al., 2001) obtained from broilers with pulmonary hypertension syndrome.

Inefficiencies of function may also occur from insufficient activity or expression of respiratory chain proteins. For instance, oxidation of respiratory chain proteins may decrease their activity and in turn the overall efficiency (coupling) of the respiratory chain. In addition, free radicals cause oxidant-mediated repression of mitochondrial transcription (Kristal et al., 1994) that exacerbates mitochondrial dysfunction by inhibiting synthesis of respiratory chain proteins (Kristal et al., 1997).

The present inventors recently reported that muscle mitochondria isolated from broiler breeder males with low feed efficiency (FE) exhibited lower respiratory control ratios, lower activities of Complex I and II (multi-protein respiratory complexes), and higher rates of hydrogen peroxide (H₂O₂) production compared to the mitochondria from broilers with high FE. (Bottje et al., 2002). Thus, feed efficiency appears to be inextricably linked to mitochondrial function and oxidative stress and the production of reactive oxygen species such as H₂O₂. Ninety percent of cellular respiration occurs in mitochondria to support ATP synthesis. However, between 1 and 4% of mitochondrial oxygen consumption is due to the generation of ROS following leakage of electrons from the respiratory chain (Chance et al., 1979). ROS generation represents inefficiency in mitochondrial function that can cause damage to critical structures; e.g. proteins, lipids, and DNA, and in so doing can precipitate more inefficiency.

In the poultry industry, companies routinely conduct feed efficiency trials on progeny of their top genetic stock in an effort to identify which progeny are most efficient in converting feed into body weight gain. In commercial poultry genetic companies, this consists of determining the amount of feed consumed by 200 to 300 birds per week. Approximately the top 5% of these birds are selected for replacement stock within the company. This process is highly labor intensive and takes at least one week to identify animals that are the most efficient within each group. Moreover, this testing is routinely performed on animals at 5-6 weeks age.

Because of the important commercial consequences of feed efficiency, it is desirable to develop a method for quickly and efficiently predicting feed efficiency. Unfortunately, the ability to predict FE in early stages of broiler development is lacking. Even at the late stage of development, such as the stage of 5-6 weeks, feed efficiency is still particularly difficult to determine quickly because it takes at least a week to gather all data. For instance, in the poultry industry, the birds and feed are weighed at the beginning of the study and again at the end. In most instances, this process takes a several weeks.

Discussion or citation of a reference herein shall not be construed as an admission that such reference is prior art to the present invention.

### 4. SUMMARY OF THE INVENTION

Therefore, this invention provides methods suitable for quickly predicting feed efficiency. More specifically, an objective of the present invention is to provide a method for determining and predicting feed efficiency by correlating mitochondrial function and biochemical factors that are associated with feed efficiency.

Further the present invention provides methods for predicting and selecting animals with feed efficiency wherein animals with or without overt high feed efficiency are identified as having predisposition for high feed efficiency by detecting the presence of protein(s) or DNA patterns associated with control samples known to possess high feed efficiency.

It is a further object to provide kits for determining if an animal has genetic predisposition for feed efficiency.

### 5. BRIEF DESCRIPTION OF THE FIGURES

FIG. 1 shows the analysis of mitochondrial function in breast and leg muscle isolated from broilers with high and low feed efficiency (FE). Functional measurements include the respiratory control ratio (RCR), the acceptor control ration (ACR), and the ADP/O ratio for breast and leg muscle mitochondria provided either A) glutamatelmalate, or B) succinate as energy sources. Values represent the mean ±SE of 6 observations (High FE, shaded bar) and 7 observations (Low FE, open bar).

* Mean value for low FE is lower than High FE mitochondria (P<0.05).

+Mean value for low FE is lower than High FE mitochondria (P<0.06).

A Mean leg muscle value for both groups is lower than breast muscle value for both groups (P<0.05).

FIG. 2 shows the analysis of mitochondrial function in liver isolated from broilers with high and low feed efficiency (FE). Functional measurements include the respiratory control ratio (RCR), the acceptor control ration (ACR), and the ADP/O ratio for liver mitochondria provided succinate as an energy source. Values represent the mean ±SE of 6 observations (High FE, shaded bar) and 7 observations (Low FE, open bar). *Mean value for low FE mitochondria is lower than High FE mitochondria (P<0.05).

FIG. 3 shows the analysis of hydrogen peroxide (H₂O₂) production (nmol/min per mg mitochondrial protein [P]) in A) breast and B) leg muscle mitochondria obtained from broilers with high (gray bars) and low (open bars) feed efficiency (FE). Mitochondria (provided glutamate as an energy substrate) were treated with no inhibitor (NI), or treated with rotenone (Rot), malonate (Mal), thenoyltrifluroacetone (TTFA), antimycin A (AA), and myxothiazol (Myx) which inhibit electron transport at Complexes (C) I, II, and III of the respiratory chain. Each bar represents the mean ±SE of 6 to 7 observations. +Mean value in the Low FE group is higher than High FE (P<0.06). * Within group treatment values for low FE group are elevated in comparison to low FE NI value (P<0.05). [symbol] Within treatment value for Rot is higher than NI value in leg muscle mitochondria (P<0.07).

FIG. 4 shows the analysis of activities (activity per min per mg mitochondrial protein) of Complex I and II in breast and leg muscle mitochondria obtained from broilers with high (gray bars) and low (open bars) feed efficiency (FE). Each bar represents the mean ±SE of 6 to 7 observations.

*Low FE values are lower than High FE values (P<0.05).

FIG. 5 shows the analysis of the relationships between feed efficiency and SA) Complex I activity, SB) Complex II activity, and SC) Complex I plus Complex II activity in breast (triangle) and leg (circle) muscle mitochondria. Regression equations shown for each relationship were significant (P<0.05).

FIG. 6 shows the analysis of the relationships between feed efficiency and the Complex II to Complex I activity ratio in breast and leg muscle mitochondria obtained from broilers with low and high FE.

FIG. 7 shows a SDS page gel electrophoresis of mitochondrial proteins obtained from breast muscle of broilers with low and high feed efficiency.

FIG. 8 shows the expression of a 47 kDa band in breast muscle mitochondria.

FIG. 9 shows the relationship of feed efficiency with relative intensity of 47 kDa protein expression.

FIG. 10 shows the expression of protein subunits of Complex III and IV in breast muscle of low and high feed broilers. Representative Western blot bands are shown above the respective bars of protein subunits. Each bar represents the mean ±SE for high FE (n=7) and low FE (n=5); cyt c1=cytochrome c1; cyt b=cytochrome b; ISP=iron-sulfur protein; COX II=cytochrome c oxidase subunit II.

FIG. 11 shows the expression of adenine nucleotide translocator I (ANTI) protein in the mitochondrial inner membrane in breast muscle of low and high fed efficient broilers. Representative Western blot bands are shown above the respective bars of protein subunits. Each bar represents the mean ±SE for high FE (n=7) and low FE (n=5). ***P≦0.003.

FIG. 12 shows analysis of 2-D gel electrophoresis indicating that there are at least 5 spots that are different between the Low and High FE mitochondria.

FIG. 13 shows electron transport chain proteins are expressed differently in liver mitochondria obtained from broilers with high and low FE.

FIG. 14 shows a comparison of proteins in plasma expressed relative to values found in high FE broilers. Bars that go up indicate proteins that are higher in the low FE broilers. Bars that go down represent values that were higher in the high FE broilers. Only proteins that were 5-fold differences between groups or greater are demonstrated.

### 6. DETAILED DESCRIPTION OF THE INVENTION

This section presents a detailed description of the invention and its applications. This description is by way of several exemplary illustrations, in increasing detail and specificity, of the general methods of this invention. These examples are non-limiting, and related variants will be apparent to one of skill in the art.

Although, for simplicity, this disclosure often makes references to broilers, poultry, etc., it will be understood by those skilled in the art that the methods of the invention are useful for the analysis of any animal. In particular, one skilled in the art will recognize that the methods of the present invention are equally applicable to other livestock, agriculturally important animals, or human health.

The description of the invention, for simplicity, is largely in terms of interaction among any number of proteins involved in mitochondria function. However, the methods of the invention are also applicable, as will be apparent to one skilled in the art, to the analysis of interactions between any two or more antibodies to a biological sample.

It should be understood that various changes and modifications to the presently preferred embodiments described herein will be apparent to those skilled in the art. Such changes and modifications can be made without departing from the spirit and scope of the present invention and without diminishing its attendant advantages.

One embodiment of the present invention provides a method for predicting or selecting feed efficiency. More specifically, this invention provides a method for predicting feed efficiency in an animal comprising (a) calculating feed efficiency of said animal comprising measuring feed intake and weight gain of said animal; (b) obtaining a biological sample from said animal; (c) calculating mitochondrial function of said animal comprising measuring oxygen consumption and electron transport activity; (d) calculating a correlation between feed efficiency and mitochondrial function comprising comparing the measured level of feed efficiency from step (a) to measured levels of mitochondrial function from step (c); and (e) predicting the likelihood of high or low feed efficiency in said animal whereby a positive or negative direct relationship between said feed efficiency and said mitochondrial function of said correlation of step (d) indicates feed efficiency.

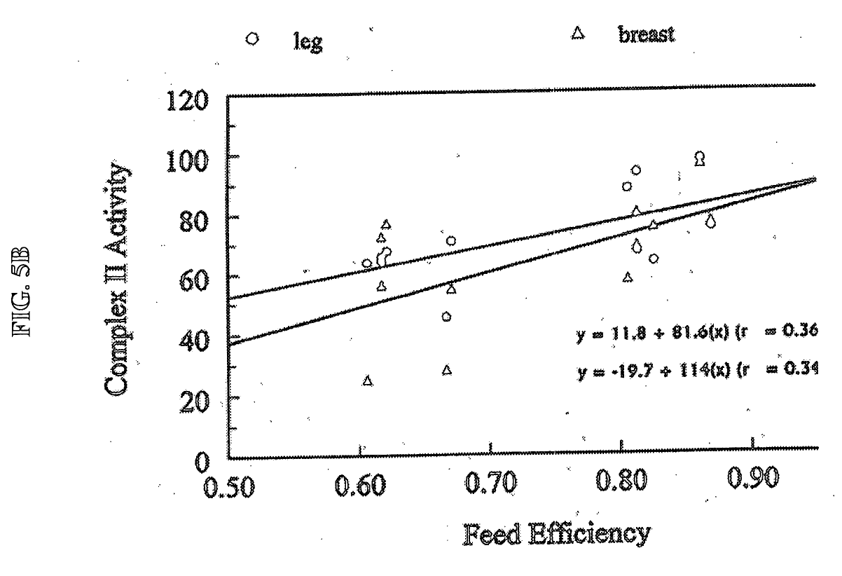

In one embodiment, mitochondrial function comprises measuring the activity of Complexes I or II or both of the electron transport system. In particular, the ratio of the activity of Complex I and II is determined and correlated to feed efficiency. Interestingly, when Complex I and II activities were combined and regressed with feed efficiency, this combined activity value improved the correlation coefficients (r²=0.41 and 0.54), and the slopes depicting complex activity and feed efficiency relationships for each muscle were nearly parallel (see FIG. 5C). Therefore, a direct positive or negative relationship between feed efficiency and mitochondrial function indicates feed efficiency.

In a preferred embodiment of the present invention, the biological sample is selected from the group consisting of blood and tissue.

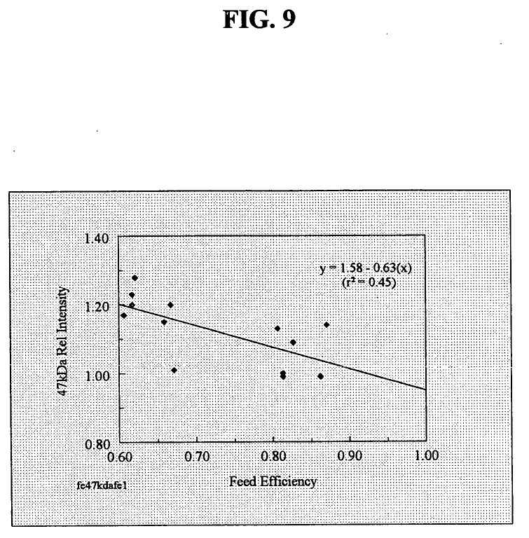

The present inventors demonstrated that there was an inverse relationship between feed efficiency and the relative intensity of a 47 kDa protein with a correlation coefficient of 0.45 (see FIG. 9). In addition to the 47 kDa protein bands, there appears to be several additional peptide bands that are differentially expressed in conjunction with feed efficiency. Therefore, another embodiment of the present invention provides a method for determining and predicting feed efficiency by correlating mitochondrial function protein patterns associated with feed efficiency. In particular, this invention provides a method for predicting feed efficiency in an animal comprising: (a) calculating feed efficiency of said animal comprising measuring feed intake and weight gain of said animal; (b) obtaining a biological sample from said animal; (c) calculating mitochondrial function of said animal comprising measuring oxygen consumption and electron transport activity; (d) calculating a correlation between feed efficiency and mitochondrial function comprising comparing the measured level of feed efficiency from step (a) to measured levels of mitochondrial function from step (c); (e) obtaining protein patterns of said biological sample; (f) comparing said protein patterns with said correlation of step (d); (g) predicting the likelihood of high or low feed efficiency in said animal, whereby a positive or negative direct relationship between said feed efficiency and said mitochondrial function of said correlation of step (d) and said protein patterns indicates feed efficiency.

In a preferred embodiment, protein patterns are analyzed on SDS polyacrylamide gel electrophoresis. The increased sensitivity of two-dimensional electrophoresis enables one skilled in the art to further identify protein candidates for biochemical probes for selection of genetic stock for commercial broilers. Therefore, in another embodiment, the present invention provides a method for identifying proteins associated with feed efficiency. In particular, protein samples are analyzed using two-dimensional gel electrophoresis wherein a direct positive or negative correlation between feed efficiency, mitochondrial function and protein patterns indicates feed efficiency whereby protein associated with feed efficiency are identified. However, one skilled in the art could use various methods for protein detection such as but not limited to high performance liquid chromatography (HPLC), fast performance liquid chromatography (FPLC), etc.

In still another embodiment, proteins associated with mitochondria function and feed efficiency are further identified by amino acid sequencing. In yet another embodiment of the present invention, monoclonal antibodies are targeted to proteins associated with feed efficiency. Therefore, the present invention provides a method for predicting feed efficiency comprising identifying proteins associated with mitochondrial function and feed efficiency comprising hybridizing said biological samples with antibodies specific for feed efficiency. The methods for preparation of both monoclonal and polyclonal antibodies are well known in the art.

In another embodiment, antibodies and revealing reagents are produced for the conduct of an immunoassay using standard detection protocols, for example radioisotope labelling, fluorescent labelling or ELISA, either in a direct or competitive format, may conveniently be supplied as kits which include the necessary components and instructions for the assay. In one embodiment of the invention such a kit includes a microtiter plate coated with a relevant synthetic peptide, standard solutions for preparation of standard curve, a urine control for quality testing of the analytical run, rabbit antibodies reactive with the above-mentioned synthetic peptide, anti-rabbit immunoglobulins conjugated to peroxidase, a substrate solution, a stopping solution, a washing buffer and an instruction manual.

Antibody types include, but are not limited to, polyclonal, monoclonal, chimeric, single chain, Fab fragments, and an Fab expression library.

In one embodiment, the kit provides a means for attaching primary and secondary antibodies to wells of a microtiter wherein a biological sample obtained from the animal is added to the wells. In a preferred embodiment, the biological sample is a blood sample. In another preferred embodiment, the sample is a homogenized tissue sample. Further, the present kit provides a method for detecting the presence of proteins associated with feed efficiency comprising obtaining a sample from the animal; adding the sample to the wells; quantifying the amount of protein bound to the antibody comprising measuring the presence or absence of a indicator. The amount of color or signal would then be proportional to the amount of protein present in the sample, which could be used as an indicator/predictor of feed efficiency.

In yet another embodiment, the kit of the present invention comprises a means for predetermining feed efficiency wherein more than one protein correlating to feed efficiency is determined. In this embodiment, the kit comprises antibodies for more than one protein. Another embodiment of the present invention provides a method for predicting feed efficiency at a much earlier age than currently being done. Therefore, this invention further provides methods for detecting and predicting feed efficiency at an early stage of development such as but not limited to obtaining samples in utero or in ovo.

In the present invention, hundreds of blood samples could be analyzed within a 24 hour period. This would cut the time for selection of animals with feed efficiency from 1 week to 1 day. Therefore, another embodiment of the present invention provides a method for determining and predicting feed efficiency within a 24 hour period.

Still further, this invention provides a method for developing an assay or series of assays that could be used as predictive measures of feed efficiency to predict feed efficiency without weighing an animal or the feed. This embodiment provides a method for rapidly selecting and predicting feed efficiency by analyzing any biological sample of body sample for proteins associated with feed efficiency. More, antibodies against proteins associated with feed efficiency would be provided in a kit of the present invention.

The present invention would be useful for identifying the biochemical and/or genetic factors responsible for feed efficiency and thereby predicting which animals are potential candidates for feed efficiency for the purposes of selection and/or providing treatment. The amount involved in selection of animals for feed efficiency would reduce dramatically. Feed and other costs in growing animals through age at selection could be greatly reduced.

Genetic gains in improving feed efficiency could be greatly accelerated thus reducing costs in producing animals. Additional benefits would include, less feed usage resulting in reduction of manure produced in animal agriculture.

The present invention further provides a method for determining and predicting genetic traits associated with feed efficiency such as but not limited to reproductive potential and genetic disorders.

Specific genes may be targeted to identify differences in nuclear and/or mitochondrial DNA variants in broilers with different feed efficiency. These findings would be helpful in developing tools of the type discussed below that could be used to aid in selecting highly feed efficient animals not just in chickens, but possibly in other animals as well. For example, the present inventor demonstrated similar feed efficiency results in broilers, cattle and swine. Further, all three groups of animals were from single genetic lines and feed identical feeds.

It is further an object of this invention to provide kits for predetermining if an animal has genetic predisposition for feed efficiency. The kit comprises a means for investigating the genotype of an animal comprising control genes or nucleic acid fragments capable of hybridizing to genes associated with feed efficiency.

In yet another embodiment, the present invention provides a kit for predicting an animal's likelihood of developing high or low feed efficiency wherein the kit comprises a means for determining genetic patterns for genes associated with feed efficiency. In particular, the kit of the present invention provides a means for determining a genetic pattern comprises a set of polymerase chain reaction (PCR) primers, which further comprises a means for collecting a DNA sample.

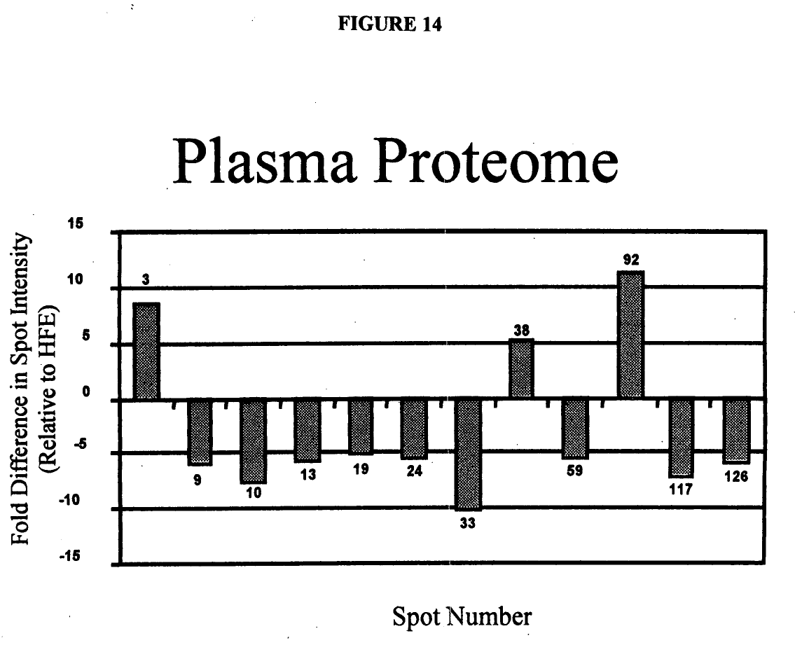

Comparison of proteins in plasma expressed relative to values found in high FE broilers revealed that feed efficiency is effective not only in the mitochondria (see FIG. 14). Therefore, the biological sample of the present invention is plasma.

Another objective of the present invention is to develop a portable biosensor for in-field detection that is a microfluidic based fluorescence immunosensor for rapid detection of specified proteins. The biosensor will utilize microfluidic immuno-channel to capture target protein and generate the fluorescence signal. In particular, a microfluidic channel is modified with antibodies specific target protein. When a sample solution flows through the antibody-modified micro-channel, the target protein will be captured on the surface and separated from other proteins. Fluorochrome-labeled antibodies are injected and sandwich immunocomplexes consisting of capture antibodies, target protein and labeled antibodies are formed. The immunocomplexes formed in the microchannel's inner surface are detected in situ with a fluorescence detector. Unlike the conventional optical or electrochemical methods that detect the products of enzyme labels, the fluorescence method directly detects the immunocomplex. No substrates are required and there is no physical contact with the sample solution and optical detector. Compared with commonly used capillary columns, microfabricated capillary channels are more attractive since the channel pattern dimensions can be defined by individual design. Further, multiple microchannels can easily be integrated into a chip. In one embodiment, a prototype biosensor for rapid detection of specific proteins is constructed by integrating an antibody-modified microfluidic channel with in-situ fluorescence measurement. This prototype provides direct and sensitive detection of the sandwich immunocomplexes of immobilized antibodies-target cell-fluorochrome labeled antibodies that are formed on the inner surface of the microfluidic channel.

The following examples are given for the purpose of illustrating various embodiments of the invention and are not meant to limit the present invention.

### 7. EXAMPLES

The invention having been described, the following examples are offered by way of illustration and not limitation.

Experiments were conducted to determine the relationships between feed efficiency (FE, feed:gain) and mitochondrial function and biochemistry. Feed efficiency was determined in a group of 100 broiler breeder males between the age of 5 and 6 weeks old. Broilers with high FE (0.83±0.01, n=6) and low FE (0.64±0.01, n=7) were selected. The differences in FE were due to greater body weight (BW) gain (P<0.05) in the high FE group, whereas feed intake did not differ between groups (P>0.50).

Breast and leg muscle mitochondria were isolated to assess mitochondrial function, electron leak, and activities of Complex I and II of the respiratory chain. The respiratory control ratio (RCR, an index of respiratory chain coupling) was higher in breast muscle and leg muscle mitochondria in the high FE group with NADH-linked energy substrates (glutamate-malate), but there was no difference in the RCR between groups with succinate, an FADH-linked energy substrate. There were also no differences in the ADP:O ratio (an index of oxidative phosphorylation) with either energy substrate between groups. The acceptor control ratio (ACR) was higher in high FE liver mitochondria and no differences in the RCR or ADP:O between groups using succinate as an energy source. Hydrogen peroxide (H₂O₂) generation (an indicator of electron leak) was higher in the low FE group, and was elevated following inhibition of Complex I and III in low FE but not high FE breast muscle mitochondria. No difference in ROS generation between FE groups was observed in leg muscle mitochondria, but Complex I inhibition (with rotenone) elevated (P<0.07) H₂O₂ generation in low FE leg muscle mitochondria.

The activities of Complex I and II were higher in high FE breast and leg muscle mitochondria compared to those in low FE mitochondria. Greater variability in the Complex II to Complex I activity ratio was observed in low FE than in high FE mitochondria for both breast and leg muscle. The results indicate that lower RCR (respiratory chain coupling) in low FE breast muscle mitochondria may be associated with higher ROS production and lower activities of Complex I and II. These findings indicate that mitochondrial function and biochemistry are associated with feed efficiency and provide insight into cellular mechanisms associated with the phenotypic expression of feed efficiency in broilers.

7.1 Materials and Methods

A. Birds and Management

Male broilers were selected from a group of 100 that were tested for feed efficiency in breeder male replacement stock (Cobb Vantress, Inc., Three Springs Farm, Okla.). At 5 wk, birds were individually housed in cages (51×51×61 cm) (Alternative Designs, Siloam Springs, Oreg. 72761) with thermoneutral temperature (25° C., 20L:4D), and feed removed for 24 hours. Calculation of feed efficiency was determined in birds from 5 to 6 weeks by measuring feed intake and body weight. From this group of birds, a total of 16 that exhibited the lowest and highest feed efficiency (FE) (n=8 per group) of the initial group of 100 were selected for this study males. The birds were color-coded, transported to the University of Arkansas, and housed in similar cages and environmental conditions. Birds were provided ad libitum access to water and the same diet during the feed efficiency trial (23.7% protein, 3,200 kcal ME).

B. Sampling Procedure

After a 5 d acclimation period, birds were randomly selected (one bird per day) from each group with group selection being alternated on successive days. Researchers at the University of Arkansas conducted these studies blind; i.e. they did not have access to the feed efficiency data for the individual birds until after the mitochondrial function studies were completed. After weighing, each bird was killed with an overdose of sodium pentobarbital by intravenous injection into the wing vein. Portions of the breast muscle (pectoralis superficialis), leg muscle (quadriceps femoris) (Chamberlain, 1943), and liver were obtained for isolation of mitochondria (see below) and a portion of each tissue immediately frozen in liquid nitrogen for biochemical analyses.

C. Mitochondrial Isolation

Breast and leg muscle mitochondria were isolated according to Bhattacharya et al. (1991), with modifications. The tissues were excised rapidly, finely minced in isolation medium A (100 mM sucrose, 10 mM EDTA, 100 mM Tris-HCl, 46 mM KCl, pH 7.4), and incubated at room temperature (25° C.) in 14 mL of isolation medium A containing 20 mg % Nagarse for 5 min. The minced tissue was homogenized and incubated for an additional 5 min on ice (4 C) with stirring. The homogenate (1,000 g for 10 min) and resulting supernatant (10,000 g for 15 min) were centrifuged to obtain the mitochondrial pellet that was resuspended and washed in 10 mL of isolation medium A plus 0.5% BSA (without Nagarase). Mitochondria were pelleted by centrifugation (8,000 g for 15 min) in incubation medium (230 mM mannitol, 70 mM sucrose, 20 nM Tris-HCl, 5 mM KH₂PO₄, pH 7.4). The resulting pellet was resuspended in 2 mL of incubation medium and placed on ice for functional and respiratory inhibitor studies described below. Liver mitochondria were isolated according to Cawthon et al. (1999; 2001).

D. Mitochondrial Function

Mitochondrial function was determined according to Estabrook (1967). Oxygen consumption of mitochondria (expressed in nmol/min per mg protein) was measured polarographically with a Clark-type oxygen electrode in duplicate 3 mL thermostatically controlled chambers equipped with magnetic stirring (Yellow Springs Instrument Co. Inc., Yellow springs, Ohio 45387) as recently described (Cawthon et. al., 1999; 2001). All duplicate measurements were averaged and completed within 3 h of isolation. Aliquots (0.5 mL) of the muscle mitochondria were removed and added to the reaction vessel containing 1 mL of RCR reaction buffer (220 mM d-mannitol, 70 mM sucrose, 2 mM HEPES, 3 mM KH₂PO₄; 5 mL of 1.5 mM rotenone, 50 μL of 1 M succinate, pH 7.0). Substrates tested in this study were either glutamate-malate (10:1 mM) or succinate (10 mM) that donate electrons to the respiratory chain at Complex I (NADH ububiquinone: oxidoreductase) and Complex II (Succinate: ubiquinone oxidoreductase), respectively. Function in liver mitochondria was determined according to Cawthon et al. (1999) using succinate (10 mM) as an energy substrate.

Indices of muscle mitochondrial function were determined according to Estabrook (1967). After monitoring the initial oxygen consumption (State 2 respiration rate), State 3 (active) respiration was initiated following the addition of 155 μM ADP (final concentration), followed by State 4 (resting respiration) when ADP levels become limiting. The acceptor control ratio (ACR) was calculated by dividing State 3 by State 2 respiration. The RCR (an index of respiratory chain coupling) was calculated as State 3 divided by State 4 respiration. The efficiency of ATP synthesis coupled to cell respiration, the ADP/O ratio, was determined by dividing the quantity of ADP added by the amount of oxygen consumed during State 3 respiration.

E. Determination of Mitochondrial H₂O₂ Production

Generation of H₂O₂ was determined using the 2′,7′-dichlorofluorescin diacetate (DCFH-DA) (Molecular Probes Inc., Eugene, Oreg. 97402) chemical probe using procedures by Iqbal et al. (2001) with modifications. H₂O₂ was measured in 96-well microplates, by a photofluorometric detector (Cytofluor 2350, Millipore Corporation, Bedford, Mass. 01730) at a sensitivity of 3 and excitation/emission wavelength at 480/530 nm, respectively. Reaction conditions for H₂O₂ measurement included the addition of 0.1 mg of mitochondrial protein, 52 μM DCFH-DA, 64 μL H₂O₂ buffer containing 145 mM KCl, 30 mM Hepes, 5 mM KH₂PO₄, 3 mM MgCl₂, 0.1 mM EGTA. Superoxide dismutase (SOD, 10 Upper well) (Sigma Chemical Co., St. Louis, Mo. 63178) was added to each well of the microplate to convert all O₂^{o-} to H₂O₂. Mitochondria were provided with pyruvate (10 mM) and malate (2 mM) as energy substrates that provide reducing equivalents to the electron transport chain at Complexes I and II, respectively. Activity remaining in wells with added catalase (225 Sigma units per well) was subtracted to account for fluorescence caused by factors other than H₂O₂ (Iqbal et al., 2001). The final volume in each well was 124 μL. The microplate was incubated at 37° C. and read sequentially at 0, 10 and 30 min by the Cytoflour photofluorimeter. Values of H₂O₂ were calculated from a standard curve with known amounts of H₂O₂. Mitochondrial protein concentration was measured by the micro protein determination kit (# 610-A)¹¹ and values of H₂O₂ expressed as nmol/min per mg of mitochondrial protein.

F. Substrate-Inhibitors Studies

Generation of H₂O₂ in lung mitochondria was monitored with and without chemical inhibitors that block electron transfer at specific sites in the respiratory chain as follows: rotenone (Complex I); 4,4,4-trifluoro-1-[2-thienyl]-1,3-butanedione (TTFA) and malonate (Complex II); myxothiazol (Complex III, Q cycle); and antimycin A (cytochrome b₅₆₂ within Complex III). Final concentrations used were rotenone (10 μM; myxothiazol (13 μM); TTFA (8 μM); antimycin A (13 μM; malonate (7 μM) under the reaction conditions mentioned above. Appropriate controls were used for all wells of the microplate, e.g., blanks for mitochondria, all inhibitors, and catalase with both substrates and final values were corrected with these blanks.

G. Complex Activity

Activities of Complex I (NADH ubuiquinone: oxidoreductase) and Complex II (Succinate: ubiquinone oxidoreductase) were assessed by ultra violet (uv) spectrophotometry. Complex I activity was measured by following the oxidation of NADH (Galante and Hatefi, 1978). Fifty microliters (μL) of mitochondria (p100 mg protein) were added to a solution containing 50 mM tris-HCl and 1.3 mM 2,6 dichloroindophenol (DCIP) in a final volume of 1 mL. The reaction was initiated with the addition of 15 mM NADH. Absorbance at 600 nm was monitored for 10 min to follow the rate of oxidation of NADH and activity determined using an extinction coefficient of ε=21 mM⁻¹ cm⁻¹. Complex II activity was determined by following the reduction of dihydroubiquinone-2 (Coenzyme Q₂) by DCIP (Hatefi and Stiggall, 1978). Mitochondria (˜100 μg protein) was added to a solution containing 74 W DCIP and 50 μM Coenzyme Q₂. The reduction of DCIP was followed at 600 nm as a function of time until about 80% of the dye (DCIP) was bleached. Enzyme activity was calculated using an extinction coefficient of ε=21 nM⁻¹ cm⁻¹. Values for Complex I and II are expressed in units of activity per min per mg mitochondrial protein.

The activity of Complex III (ubiquinol: ferricytochrome c oxidoreductase) was measured by the rate of reduction of cytochrome c by uiquinol-2 (Hatefi, 1978). Mitochondria (˜40 μg protein) were added to a solution containing 35 mM KH₂ PO₄, 5 mM MgCl₂, 3 M KCN, 0.25% BSA, 15 W cytochrome c (III), and 1 μM rotenone in a final volume of 1 mL. The reduction of cytochrome was measured at 550 nm for 10 min. The enzyme activity was calculated with an extinction coefficient factor (c=19.2 mM⁻¹cm⁻¹).

The activity of Complex IV (ferrocytochrome c: oxidoreductase) was measured according to Galante and Hatefi (1978) and was carried out by evaluating the oxidation of cytochrome c as a decrease in absorbance at 550 nm. Cytochrome c was reduced by adding 50 μM to 0.1 M dithionite containing 10 mM Tris-HCl. The reaction mixture contained 35 mM KH₂PO₄, 5 mM MgCl₂, 3 mM KCN, 0.25% BSA, 15 mM reduced cytochrome c, 1 μM rotenone to a final volume of 1 mL. The reaction was initiated by adding mitochondria (˜40 μg protein) and the oxidation was followed at 550 nm for 10 min. The activity was calculated with an extinction coefficient factor (ε=19.2 mM⁻¹ cm⁻¹)

H. Statistical Analyses

Data are presented as the mean ±SEM and means separated by t-tests. Regression analysis was accomplished using JMP In® statistical analyses software (SAS Institute Inc., Cry, N.C.). A probability level of P≦0.05 was considered statistically significant.

I. Two-Dimensional Gel Electrophoresis

The first dimension was performed utilizing Immobiline DryStrips (pH 3-10 NL, 24 cm) using the Multiphor II isoelectric focusing system (Amersham Pharmacia Biotech). Whole breast muscle or mitochondria (50 μg) was diluted in rehydration buffer (8 M urea, 2% CHAPS, 2% v/v IPG (3-10 NL), 0.33 mg/ml dithiothreitol, and trace bromphenol blue). Two-dimensional PAGE was performed following a modified version of O'Farrell's method (O'Farrell, 1975). Briefly, the first dimension is isoelectric focused with Immobiline DryStrip immobilized pH 3-10 non-linear immobilized gradient gels from Amersham Pharmacia Biotech. Isoelectric focusing was performed at constant volts using a Multiphor II for 45-60 kVolt hours. The IPG strips is equilibrated in SDS-PAGE sample buffer and the second dimension is performed with 12.5% acrylamide as previously described (Pumford et al., 1990) using an Ettan DALT system (Amersham Pharmacia Biotech). Proteins on the gels are visualized using a silver stain (Amersham Pharmacia Biotech). Gels are digitalized using an Agfa Arcus II densitometer and the image is analyzed using ImageMaster 2D Elite software (Amersham Pharmacia Biotech).

J. Blue Native Electrophoresis

Mitochondrial proteins are extracted for 20 minutes in 0.75 M aminocaproic acid with 50 mM Bis Tris and centrifigued for 10 minutes at 14,000 g. The supernatant is mixed with a final concentration of Coomassie brilliant blue g-250 (0.02% w/v) in 0.5 M aminocaproic acid. The protein is separated using a 5-12% polyacrlamide blue native el wit Bis Tris (50 mM) anode buffer and Tricine (50 mM), Bis Tris (15 mM), Coomassie brilliant blue G-250 (0.02% w/v) cathode buffer. Lanes form the blue native gel are transferred to a second dimension denaturing 12% SDS-PAGE using modifications for the transition from first to second dimension. Gel staining and spot identification will as described above.

K. In-gel Trypsin Digestion

Silver-stained gels are destained using 15 mM potassium ferricyanide and 50 mM sodium thiosulfate. Gels were digested with trypsin by the improved method of Katayama (Katayama et al., 2001). Briefly, gels are washed five times with 50 μl of 50% methanol/40% water/10% acetic acid for 5-20 min. The gels are mixed with 500 μl of 50 mM ammonium bicarbonate solution for 5 min, and then with 500 μl of acetonitril for 5 min., then dried in a Speedvac evaporator. The gel is incubated for 5-10 min. in 2 μl of 25 mM ammonium bicarbonate containing 0.05 μg of trypsin and 0.1% n-octyl glucoside. Then 10 μl of 25 mM ammonium bicarbonate containing 0.1% n-octyl glucoside is added and the mixture is incubated at 37° C. for 2 hr. The tryptic peptides are extracted twice with 40 μl of acetonitril/water/trifluoroacetic acid (66:33:0.01, v/v/v) solution in a 350W sonicator for 10 min. The extracts are dried with a Speedvac evaporator.

L. Peptide Molecular Mass Fingerprinting

The dried extracts are redissolved in acetonitril/water/trifluoroacetic acid (5:95:0.1, v/v/v). Peptide molecular mass analysis is performed by matrix-assisted laser desorption/ionization time-of-flight mass spectrometry (MALDI-TOF) with a Bruker Reflex III (Bruker Daltonics Inc., Billerica, Mass.). The samples were analyzed by the Statewide Mass Spectrometry Contract Facility housed in the Department of Chemistry and Biochemistry at the University of Arkansas and run by Dr. Richard Fleming.

M. SDS-PAGE and Western Blots

Mini SDS-PAGE and Western blots were used for the detection of mitochondrial protein oxidation (carbonyl) and respiratory chain subunits expression using isolated mitochondria and homogenate of breast muscle, respectively. Proteins were separated with SDS-PAGE on 10% gels using the methods described by Laemmli (1970) and proteins were transferred either onto nitrocellulose (0.45 μm) or polyvinylidene difluoride (PVDF) membranes in a submerged system using Hoefer™ transfer units (Pumford et al., 1990). For SDS-PAGE, proteins were separated with a 10% polyacrylamide gel in Tris-HCl buffer using a Hoefer™ electrophoresis mini-gel system at 100 V for 50-60 min. After electrophoresis, gels were soaked in transfer buffer (120 mM glycine, 15 mM Tris, and 20% (v/v) methanol, pH 8.3) and electroblotted onto the membranes (nitrocellulose or PVDF) at 80 V overnight and 100 V for one hour in the morning (Pumford et al., 1990).

N. Immunoblots for Mitochondrial Proteins.

Blots were developed using specific primary antibodies for respiratory chain protein subunits and protein carbonyls using a peroxidase-based chemiluminescence detection system. Blocked blots were incubated at room temperature for 1 to 2 hours or overnight with primary antibodies diluted in buffer containing 0.5% casein, 150 mM NaCl, 10 mM Tris and 0.02% Sodium ethylmercurithiosalicylate (Thimersal, pH 7.6). Primary antibody for adenine nucleotide translocator (ANT1) (VWR Scientific Products Co.) was used in the dilution of 1:300. After incubation once with primary antibodies, blots were washed with detergent buffer (0.5% casein, 150 mM NaCl, 1.0 mM Tris 0.02% Thimersal, 0.1% SDS, 5% Triton X-100) and twice with washing buffer (0.5% casein, 150 mM NaCl, 10 mM Tris 0.02% Thimersal) for 5 min each and rinsed with distilled deionized H₂O (three times) before and after incubation with detergent or washing buffers. Blots were incubated for 90 min with the appropriate peroxidase labeled secondary antibodies (goat anti-mouse IgG or rabbit antisera). After washing the blots extensively with detergent, washing buffers, and Tris-saline, they were treated with substrate (SuperSignal® West Dura Extended Duration) for 5 min and chemiluminescence bands were detected using a charge-coupled device (CCD) camera (Fuji LAS 1000plus) The molecular weights of separated proteins were estimated by comparison with ProSieve color molecular weight standards (Molecular Probes, Eugene, Oreg.). Mouse anti-glyceraldehyde-3 phosphate dehydrogenase (GADPH) or glutamate dehydrogenase (GDH) antibodies were used as internal standards. Bands were quantified using Scion™ software.

O. Immunoblots for Protein Carbonyl.

Protein carbonyl was determined using a reaction of dinitrophenyl hydrazine (DNP) with carbonyls (aldehydes and ketones) on proteins by methods described by Keller et al. (1993) with modifications. Carbonyl formation on proteins was detected in a Western blot format, proteins were separated by SDS-PAGE and then transferred onto PVDF membranes and incubated in one volume of 20 mM 2,4-DNP in 10% (v/v) trifluoroacetic acid and two volumes of 12% SDS. After 15 min, 1.5 volumes of 2 M Tris-base was added and incubated for 20 min. Blots were developed as described above for Western blots using anti-dinitrophenyl antiserum (1:1,000 dilution) with a peroxidase based chemiluminescence assay.

P. Protein sanding Patterns.

Protein proteins were separated using SDS-PAGE and stained with Commassie blue (R-250) for 1 h. The gels were destained with methanol-acetic acid to get the appropriate contrast, scanned for intensity analysis using Agfa (Arcus II) scanner (Agfa-Gavaert, Nev.). Protein band intensity was quantitated using Scion software™.

### 7.2 Example I

Growth Performance

Growth performance data of broilers that were utilized in this study are provided below in Table 1.

TABLE 1
Growth performance data for broilers with low and high feed
efficiency (FE)¹

| Variable | High FE (n = 6) | Low FE (n = 7) | P value |
|---|---|---|---|
| 5 Wk BW (g) | 2390 ± 38 | 2376 ± 10 | 0.702 |
| 6 Wk BW (g) | 3324 ± 56 | 3111 ± 66 | 0.043 |
| Gain (g) | 935 ± 42 | 735 ± 65 | 0.041 |
| Feed (g) | 1134 ± 61 | 11347 ± 85 | 0.911 |
| FE (g feed/g gain) | 0.83 ± 0.01 | 0.64 ± 0.01 | <0.0001 |
| FCR² (g gain/g feed) | 1.21 ± 0.02 | 1.57 ± 0.03 | <0.0001 |

¹Values are mean ± SE of values shown in parentheses.
²Feed conversion ration.

Successful mitochondrial function studies were conducted on 6 and 7 birds in the low and high FE groups, respectively from a total of 8 birds per group. At the age of 5 weeks, the initial body weight was not different but the high FE group were heavier at 6 weeks due to faster growth rate as there were no differences in feed intake (P=0.91) between groups. Table 1 demonstrates that feed efficiency (FE, g feed/g gain) was 0.64±0.01 and 0.83±0.01 for low and high FE groups, respectively. Feed conversion ratios (FCR, g gain/g feed) for each group are also shown in Table 1.

### 7.3 Example II

Mitochondrial Protein and Oxygen Consumption

Table 2 provides data for breast and leg muscle mitochondrial protein and respiration rates for high and low FE birds. There were no differences in protein levels in mitochondrial isolates between groups. Further, there were no differences between high and low FE mitochondrial respiration for state 2 (prior to ADP addition), state 3 (active respiration in the presence of excess ADP), or state 4 (resting respiration when ADP becomes limiting) in muscle or liver mitochondria. State 2 respiration was higher in leg muscle than in breast muscle in the high FE group when glutamate-malate was used as an energy source. Leg muscle respiration (State 2, 3, and 4) was higher in the high FE group than in breast muscle mitochondria when succinate was provided as an energy source. There were no differences in respiration rates between leg and breast muscle mitochondria in the low FE group when either glutamate-malate or succinate was provided as an energy source. Muscle mitochondria treated with succinate exhibited higher respiration rates compared to liver mitochondria in both high and low FE birds.

TABLE 2
Mitochondrial protein and oxygen consumption (State 2, 3, and 4
respiration) in breast and leg muscle mitochondria (provided
glutamate-malate or succinate as energy substrate) and in
liver mitochondria (provided succinate as an energy substrate)
isolated from broilers with high and low feed efficiency (FE)

| | | | | | |
|---|---|---|---|---|---|
| | High FE | Low FE | | High FE | Low FE |
| Variable | (n = 6) | (n = 7) | Variable | (n = 6) | (n = 7) |

Protein (mg/mL)

| | | | | | |
|---|---|---|---|---|---|
| Breast | 3.2 ± 0.4 | 2.5 ± 0.3 | — | — | — |
| Muscle | | | | | |
| Leg | 3.1 ± 0.4 | 2.6 ± 0.2 | — | — | — |
| Muscle | | | | | |
| Liver | — | — | — | — | — |

Oxygen Consumption (nanoatoms of O/min per mg protein)

| Breast Muscle (glutamate-malate) | | | Breast Muscle (succinate) | | |
|---|---|---|---|---|---|
| State 2 | 18 ± 2 | 22 ± 3 | State 2 | 43 ± 10 | 53 ± 9 |
| respiration | | | respiration | | |
| State 3 | 129 ± 23 | 119 ± 17 | State 3 | 87 ± 17 | 109 ± 16 |
| respiration | | | respiration | | |
| State 4 | 16 ± 3 | 20 ± 2 | State 4 | 38 ± 9 | 49 ± 8 |
| respiration | | | respiration | | |
| Leg Muscle (glutamate-malate) | | | Leg Muscle (succinate) | | |
| State 2 | 24 ± 2* | 24 ± 2 | State 2 | 65 ± 8* | 76 ± 8 |
| respiration | | | respiration | | |
| State 3 | 132 ± 13 | 112 ± 14 | State 3 | 135 ± 15* | 146 ± 13 |
| respiration | | | respiration | | |
| State 4 | 16 ± 1 | 19 ± 2 | State 4 | 60 ± 7* | 69 ± 6 |
| respiration | | | respiration | | |

Liver (with succinate)

| | | | | | |
|---|---|---|---|---|---|
| — | — | — | State 2 | 17 ± 1^{A} | 19 ± 1A |
| | | | respiration | | |
| — | — | — | State 3 | 75 ± 5^{A} | 71 ± 6A |
| | | | respiration | | |
| — | — | — | State 4 | 19 ± 1^{A} | 20 ± 1A |
| | | | respiration | | |

¹Values represent the mean ± SE.
*Within group respiration values for leg muscle are higher than breast muscle (P < 0.05).
^{A}Liver mitochondrial respiration rates (State 2, 3, and 4) are lower than in breast and leg muscle mitochondria (P < 0.05).

### 7.4 Example III

Assessment of Mitochondrial Function in Broilers with High and Low Feed Efficiency

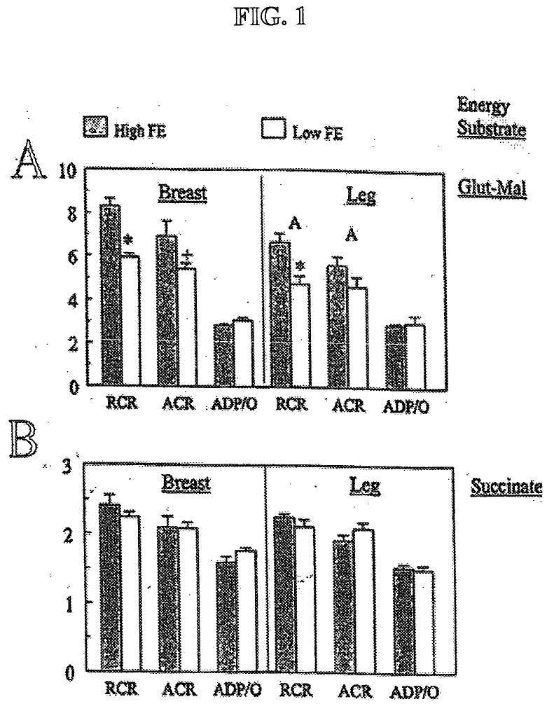

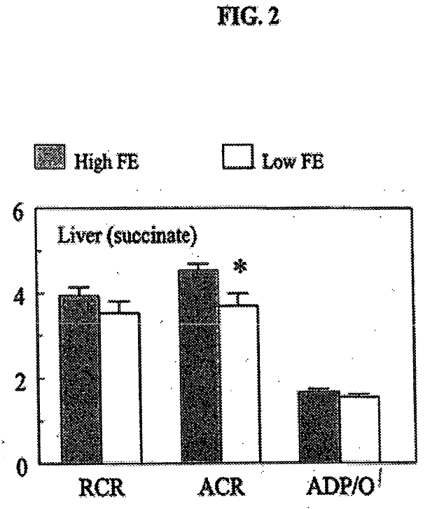

RCR (state 3/state 4) was higher (P<0.01) in both breast and leg muscle mitochondria in the high FE group (see FIG. 1A) when muscle mitochondria were treated glutamate-malate (which provides electrons to the transport chain at Complex I). These results indicate that electron transport was more tightly coupled in high FE than in low FE muscle mitochondria. Regression analysis revealed that breast mitochondria RCR values were highly correlated with feed efficiency (y=11.3(FE)-1.20, r²=0.72, P<0.001). Leg muscle mitochondria RCR values were also correlated with feed efficiency (y=7.9(FE)-0.14, r²=0.37, P<0.01). Marginally higher ACR values (P<0.06) were also observed in high FE breast muscle mitochondria provided glutamate-malate. There were no differences in mitochondrial function in muscle mitochondria provided succinate (without rotenone) obtained from high and low FE birds (see FIG. 1B). These findings provide evidence that functional differences (i.e. differences in electron transport chain (ETC) coupling) between the two groups might be due to differences associated with electron transport within Complex I. There were also no differences in the ADP:O with either energy substrate indicating that there were no apparent differences in the ability of the leg and breast muscle to carry out oxidative phosphorylation between high and low FE birds. In the liver, high FE mitochondria provided succinate exhibited a higher ACR than did low FE mitochondria, but there were no differences in the RCR or ADP:O between groups (see FIG. 2).

Leg muscle mitochondria isolated from high FE broiler exhibited higher respiration rates than did breast muscle mitochondria when succinate was provided as an energy source. Surprisingly, similar findings were not observed in low FE mitochondria (see Table 2). Yet, higher respiratory chain coupling (RCR values) was observed between low and high FE muscle mitochondria (see FIG. 1A) with glutamate-malate but not with succinate (see FIG. 1B). What role the difference in respiration rates between leg and breast muscle mitochondria in high FE birds, but not in low FE birds, contributes to mitochondrial function or feed efficiency is not apparent.

The higher RCR values in breast muscle compared to leg muscle (see FIG. 1) concurs with findings in rabbit muscle mitochondria by Youlanda and Blanchard (1970). State 3 respiration rates with Complex I (NADH-linked substrates) were higher in red muscle than white muscle in the rabbit (Jackman and Willis, 1996). There were no differences in State 3 respiration between leg (red) and breast (white) muscle in either low or high FE birds metabolizing glutamate-malate (NADH-linked substrate) (see Table 2). However, respiration rates were higher in FE leg muscle mitochondria than in breast muscle provided succinate, but differences in respiration rate were not observed between muscles in low FE mitochondria (see Table 2).

7.5 Example IV

Assessment of Electron Leakage in Muscle Mitochondria from Broilers with High and Low Feed Efficiency

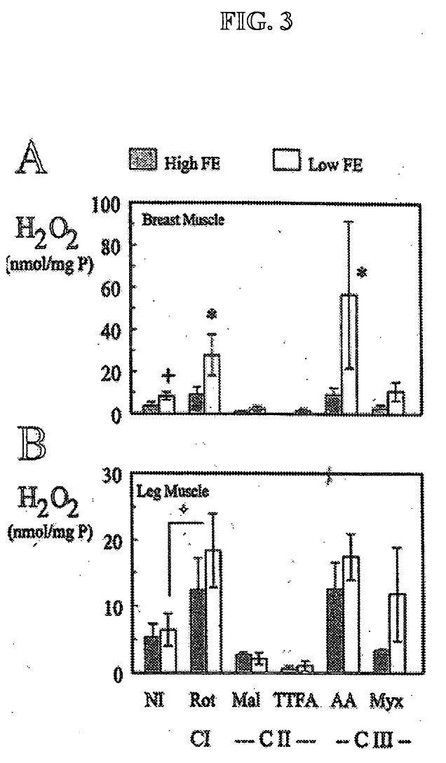

H₂O₂ production in breast and leg muscle mitochondria was determined with and without various inhibitors of the electron transport chain (see FIG. 3) to determine the relationship of lower RCR values in low FE muscle mitochondria with increased electron leak from the respiratory chain. Basal H₂O₂ production (no inhibition, NI) was greater (P<0.06) in low FE than in high FE breast muscle mitochondria (see FIG. 3A). Inhibiting electron transport at Complex I with rotenone (Rot) and Complex III (cytochrome B₅₆₂) with antimycin A (AA) raised H₂O₂ production, and therefore electron leak, in low FE but not in high FE breast muscle mitochondria. No differences were observed when electron transport was inhibited at Complex II (with malonate and TTFA) or the Q cycle of Complex III (with myxothiazol). These findings indicate that low FE mitochondria exhibit greater electron leak than high FE breast mitochondria and that this leak may be due to defects in electron transport within Complex I and III (cytochrome b₅₆₂). There were no differences in H₂O₂ production between groups with any inhibitor treatment in leg muscle mitochondria. However, an elevation (P<0.07) observed in the low FE group following treatment with rotenone (see FIG. 3B) indicates this could be a potential site of electron leak in low FE leg muscle mitochondria. In addition, there were no differences in H₂O₂ production observed in isolated liver mitochondria between groups (data not shown).

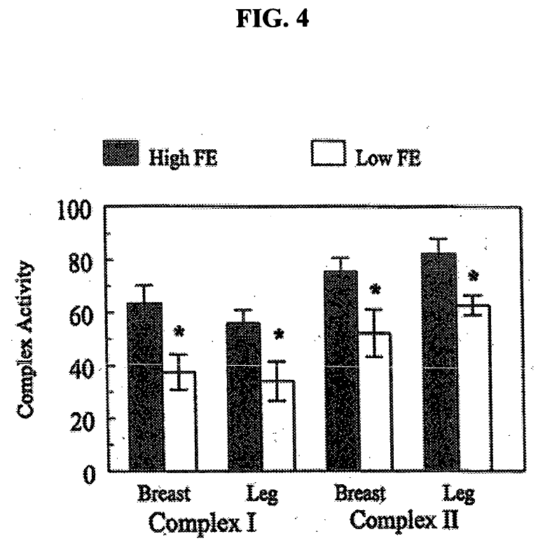

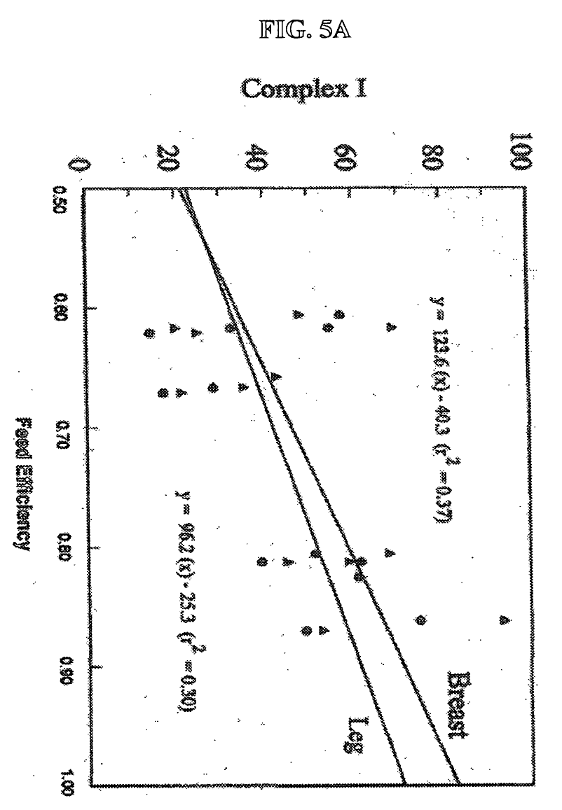

The present invention demonstrates for the first time mitochondrial function in predominantly red (leg) and white (breast) muscle fibers in poultry. Studies by Hoppeler et al. using electron microscopy revealed higher mitochondrial content in red fibers than in white (1987). This might account for the higher aerobic capacity in red versus white muscle fibers in mammals (Baldwin et al., 1972). Jackman and Willis (1996) reported that the gracillis muscle (white fiber type) exhibited 50% of maximal activity of several inner mitochondrial membrane proteins compared to soleus (red fiber type) in rabbits, suggesting that there is roughly one-half the enzymatic protein of the respiratory chain in white muscle mitochondria. These results differ somewhat from the present study in chickens in which there was little difference in Complex I and II activity between breast and leg muscle mitochondria (within either the high or low FE groups, see FIG. 4). The differences in results between the present study and that of Jackman and Willis (1996) are not apparent but could be due to species or in how activity measurements were obtained between the studies. In the present invention, respiratory chain complex activities were measured by ultra violet spectroscopy in from mitochondria after a single freeze thaw procedure. Jackman and Willis (1996) measured maximal respiration rates polarographically of respiratory chain components following repeated (5 times) sonication and freeze thaw procedures. However, using the same logic as Jackman and Willis (1996), the results of the complex activity measurements would suggest that low FE muscle mitochondria exhibit 20 to 40% lower expression of inner mitochondrial membrane proteins compared to high FE mitochondria (see FIG. 4). Caution should be used when equating enzyme activity with protein expression. Nonetheless, the fact that enzyme activity of both mitochondrial and whole tissue (above) are lower in low FE tissue suggests that the phenotypic expression of low FE is associated with lower expression of key proteins associated with mitochondrial function and antioxidant activity.

### 7.6 Example V

Assessment of Differences in Respiratory Chain Complex Activity in Mitochondria from Broilers with High and Low Feed Efficiency

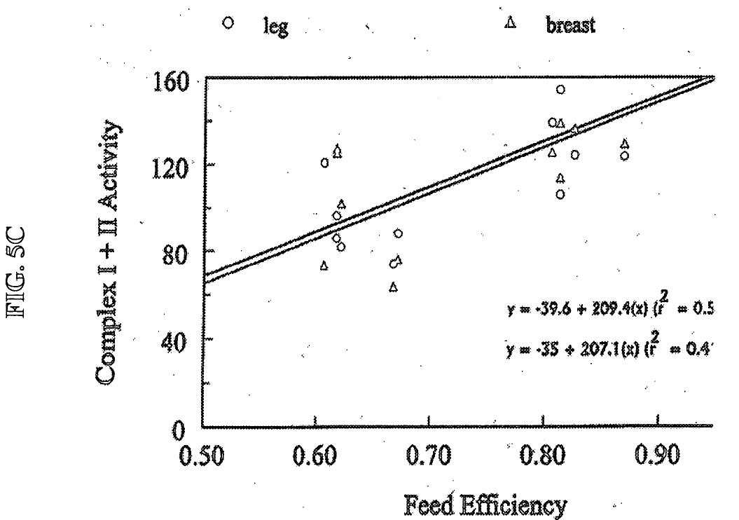

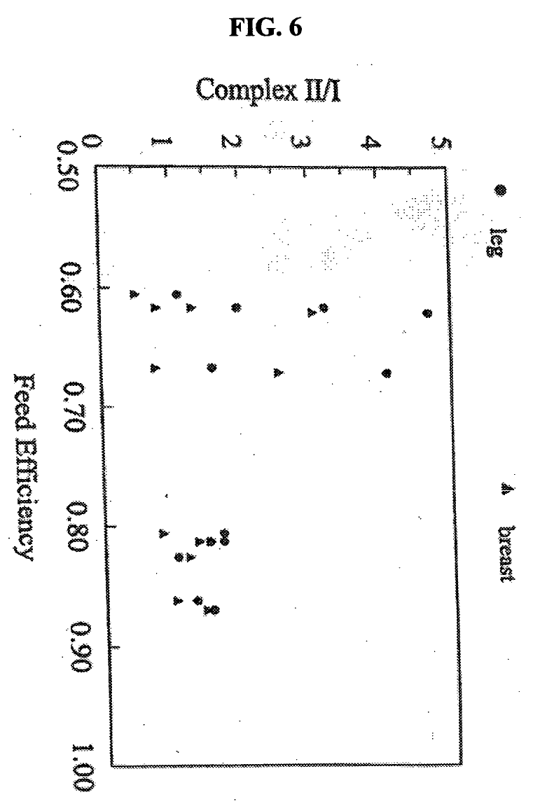

Activities of NADH-linked (Complex I) and FADH-linked (Complex II) were assessed in breast and leg muscle mitochondria. Both Complex I and II activity were lower in low FE than in high FE muscle mitochondria (see FIG. 4). The regression equations shown for Complex I activity (see FIG. 5A) and Complex II (see FIG. 5B) were all significant (P<0.05) and positively correlated (r² values ranging from 0.30 to 0.37) with feed efficiency for both muscle types. Interestingly, when Complex I and II activities were combined and regressed with feed efficiency, this combined activity value improved the correlation coefficients (r²=0.41 and 0.54), and the slopes depicting complex activity and feed efficiency relationships for each muscle were nearly parallel (see FIG. 5C). Possibly, higher correlations with feed efficiency might be achieved by adding activities of the remaining complexes (Complex III, IV and V) in future studies. The relationship in the Complex II to Complex I (CII:CI) activity ratio indicates that a much greater variation in relative activities was observed in low FE breast and leg muscle mitochondria than was observed in nigh FE muscle mitochondria (see FIG. 6).

The ratio of Complex II to Complex I (CII:CI) activity provides additional insight regarding relationships between mitochondrial function and feed efficiency. In particular, low FE mitochondria exhibited a much greater variation in the CII:CI ratio than did high FE mitochondria (see FIG. 6). This finding is particularly interesting because Complex I and II accept electrons from different energy substrates. Thus, a more balanced activity ratio for Complex I and II may be needed for efficient mitochondrial function, cell function, and in turn, for greater feed efficiency in broilers.

Combined activities of Complex I, II, III and IV from same sample sets that were regressed with feed efficiency revealed a correlation coefficient of r²=0.57. Complex III and IV revealed correlation coefficients of 0.60 and 51, respectively. Interestingly, Complex III demonstrated the highest complex activity correlation for mitochondrial functional to feed efficiency.

The results of the present invention provide the first evidence that mitochondrial function is inextricably linked to feed efficiency in any agriculturally relevant species. Muscle mitochondria from broiler males designated as having low FE exhibited lower RCR values (see FIG. 1A) (suggesting a decrease in respiratory chain coupling), higher electron leak from the respiratory chain (breast muscle only) (see FIG. 3A), and lower activities of Complex I and II of the respiratory chain (see FIG. 4). It should be noted that birds designated as low FE in this study (see Table 1) would be considered as being quite superior in feed efficiency when compared to commercial broiler production, even accounting for differences between laboratory and field conditions. Thus, mitochondrial function detected in the low and high FE groups in this study actually reflect differences within groups of very efficient birds. Even more dramatic differences might be obtained if a greater range of feed efficiencies had been examined. It should also be noted that the results of the in vitro functional studies provide only a single snapshot of what may be continuously occurring in vivo. Greater differences in mitochondrial function between low and high FE groups would likely be obtained over prolonged periods of time. The possibility of accumulative effects of enhanced peroxide production and lower respiratory chain coupling could be contributing to the phenotypic expression of feed efficiency between the groups of birds in this study.

### 7.7 Example VI

Differences in Protein Expression Associated with Feed Efficiency

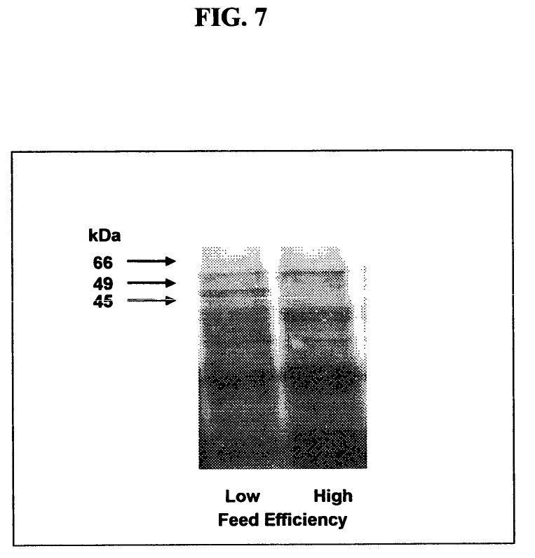

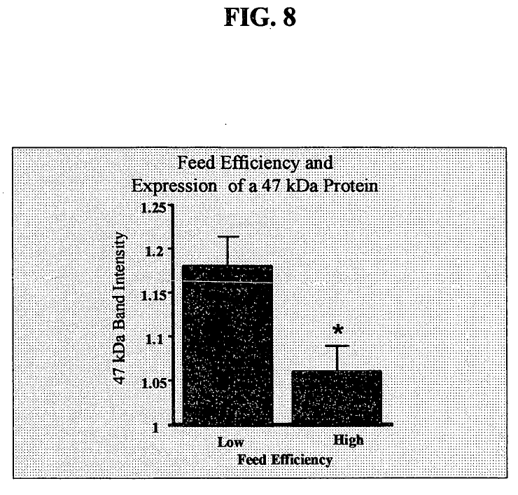

Studies using one-dimensional SDS gel electrophoresis separating mitochondrial proteins isolated from breast muscle of broilers with low and high feed efficiency revealed an ˜47 kilodalton (kDa) protein (see FIG. 7) with an increased expression (P<0.05) in low FE compared to mitochondria from high FE broilers (see FIG. 8). The expression of a 47 kDa band in breast muscle mitochondria was higher in broiler breeder males with low feed efficiency (n=7) than in broiler breeder males with high feed efficiency (n=6) (P<0.05). Moreover, the regression equation and r value shown were significant (P<0.01).

### 7.8 Example VII

Identification of Proteins Differentially Expressed with Feed Efficiency

There was an inverse relationship between feed efficiency and the relative intensity of the 47 kDa protein with a correlation coefficient of 0.45 (see FIG. 9). In addition to the 47 kDa protein bands, there appears to be several additional peptide bands that are differentially expressed in conjunction with feed efficiency. Since the low feed efficient mitochondria exhibited greater levels of oxygen radical production than did mitochondria from broilers with high feed efficiency (Bottje et al., 2002), it is possible that some of these proteins are upregulated in response to oxidative stress. Mitsumoto reported that as many as 40 polypeptides were upregulated by low-level oxidative stress (Mitsumoto et al., 2002).

The expression of proteins of the respiratory chain is under dual genetic control by both nuclear and mitochondrial DNA (Sue and Schon, 2000). Mitochondrial DNA encodes 22 tRNA's, 2 rRNA's, and 13 proteins that are all subunits of various respiratory chain complexes (Anderson et al., 1981; Desjardin and Morais, 1990). The genome is found in every nucleated cell with 2 to 10 copies per mitochondrion and as many as 800 mitochondria (e.g. in hepatocytes) can be present within a cell Robin and Wang, 1988). Free radicals cause oxidant-mediated repression of mitochondrial transcription (Kristal et al., 1994) that exacerbates mitochondrial dysfunction by inhibiting synthesis of respirator; chain proteins (Kristal et al., 1997). The proximity of the respiratory chain to the relatively unprotected mitochondrial DNA and accessory proteins required for transcription makes mitochondrial transcription vulnerable to oxidative stress (Kristal et al., 1994). Increased radical production and dysfunction (lower RCR and ADP:O) has been observed in several tissues in broilers with pulmonary hypertension syndrome (Cawthon et al., 2001; Iqbal et al., 2001; Tang et al., 2001). Thus, it is possible that the decrease in respiratory chain coupling (the RCR) in low FE breast muscle mitochondria (FIG. 1A) could be linked to oxidative damage of mitochondrial DNA or mitochondrial proteins as a result of increased electron leak and oxygen radical production (see FIG. 3A). Although electron leak was not as prominent in leg muscle mitochondria as in breast muscle mitochondria in low FE birds (see FIG. 3), the activity of both Complex I and II were lower in both breast and leg muscle mitochondria in low FE birds (see FIG. 4). Low FE muscle may also exhibit lower expression of other cellular proteins as glutathione peroxidase activity was also approximately 50% lower in low FE whole breast muscle tissue homogenate (unpublished observations). Without ruling out the possibility of oxidative damage of mtDNA or post-translational modification of proteins in Complex I and II in low FE mitochondria, these findings point towards an inherent difference in genetic expression of respiratory chain proteins in low and high FE muscle mitochondria, as the synthesis of the 4 proteins associated with Complex II (succinate:ubiquinone oxidoreductase) is controlled entirely by the nuclear genome (Sue and Schon, 2000).

The present invention provides evidence that mitochondrial function, respiratory chain activity, and electron leak are linked to feed efficiency in broiler breeder males identified as having low or high feed efficiency.

### 7.9 Example VIII

Post-Modification Analyses of Low FE Mitochondria

Detection of protein carbonyls and Western blot analysis revealed higher protein carbonyl levels (protein oxidation) in muscle mitochondria from low FE broilers than in high FE broilers. When protein carbonyl levels were regressed with Complex I activity, a negative correlation (R=0.75, P<0.01) was observed. One possible explanation is an increased in oxidative damage of electron transport chain proteins.

### 7.10 Example IX

Analysis of Protein Carbonyls in Low and High FE

A study to determine if oxidation of proteins occurs in an intact ETC complex using blue native electrophoresis (Schagger and von Jagow) revealed increased oxidation of Complex III proteins in breast muscle obtained from broilers with low FE compared to samples form broilers with high FE. Moreover, addition of Ubiquitin (Ub), a highly conserved 76 amino acid polypeptide leads to degradation of proteins by preoteasomes. Tissues probed with an ubiquitin antibody demonstrated an increase in UB (P>0.01) levels in low FE compared to high FE broilers in breast homogenate.

### 7.11 Example X

Assessment of Electron Transport Chain Protein Expression

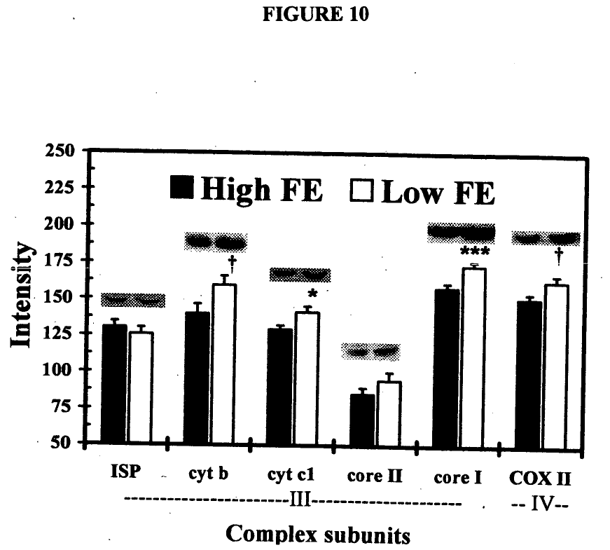

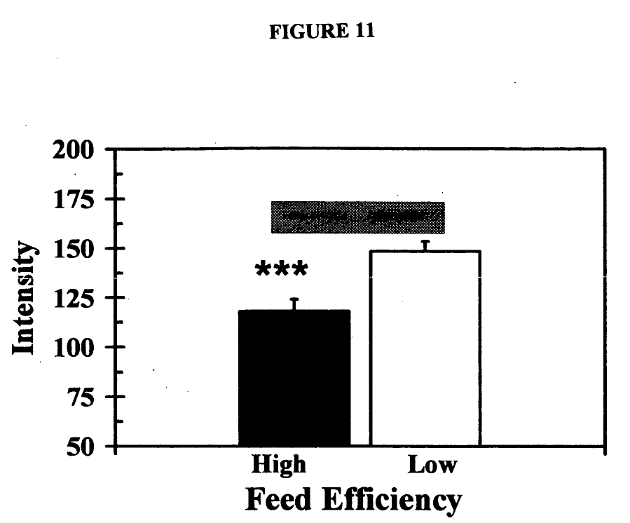

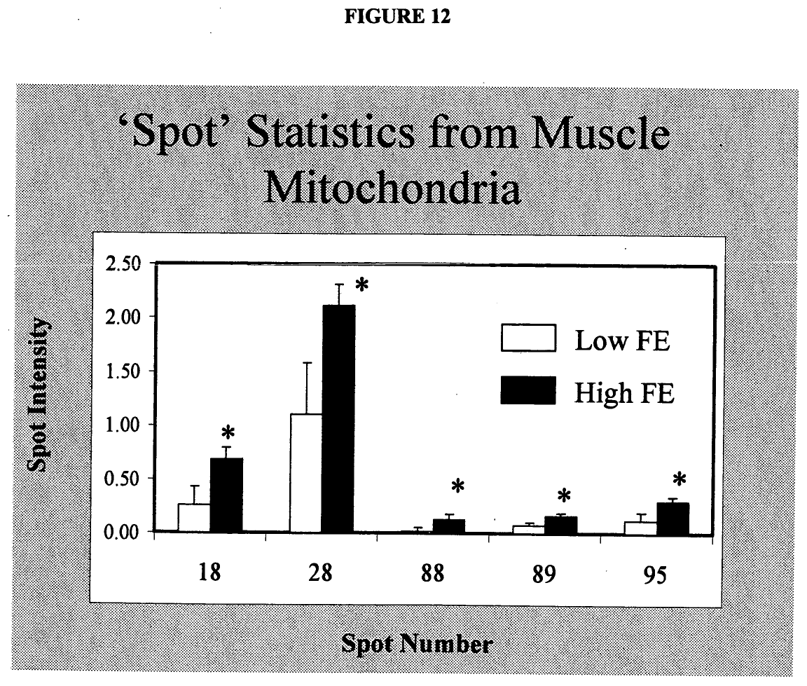

Muscle mitochondrial from low and high FE broilers were probed with antibodies for specific proteins subunits and determined by Western analysis for expression. The results indicated that the expression of cyt b, cyt c1 and core I proteins (subunits of Complex III) were higher in low FE mitochondrial. There was also a higher expression of the cyctochrome oxidase subunit II (COX II) in low FE mitochondria. Cox II and core I proteins were expressed at higher levels in the neck muscle of the high FE compared to low FE cattle (see FIG. 10). Moreover, 2-dimensional electrophoresis analysis revealed at least 6 peptides that are differentially expressed (P>0.05) between high and low FE muscle mitochondria (see FIG. 12). Further, there is evidence of other proteins that are differentially expressed in low and high FE tissue. For example, the adenine nucleotide translocator I (ANTI) in the inner mitochondrial membrane was significantly higher in low FE compared to high FE broilers (see FIG. 11).

### 7.12 Example XI

Lower Electron Transport Chain Complex Activities in Low FE Mitochondria.

Assessment of ETC Complexes I, II, III and IV resulted in suppression of all complex activities in broiler with low FE. Moreover, Complex I activity of mitochondria isolated from low FE swine muscle was significantly reduced. Regression analysis revealed that in broiler Complex III activity exhibited the highest correlation (r²=0.60) with FE of any of the ETC complexes. The complex activities were measured by a spectrophotometer, while the levels of oxidized protein (carbonyl) and immunoreactive mitochondrial proteins were analyzed using Western blot analysis. Protein carbonyl levels were higher in low FE compared to high FE broilers breast muscle indicating enhanced protein oxidation in low FE mitochondria. Activities of all respiratory chain complexes (I, II, III, IV) were higher in high Fe compared to low FE broilers for breast mitochondria. The expression of immunoreactive proteins was higher in low FE muscle mitochondria for five mitochondrial proteins; core I, cyt c1, cyt b (Complex III), COX II (cytochrome c oxidase subunit II, Complex IV) and adenine nucleotide translocator (ANTI) but there were no differences between groups in the expression of nine other respiratory chain protein subunits associated with Complex I, II, III, IV and V.

### 7.13 Eample XII

Assessment of Protein Subunits in the Respiratory Chain Complex

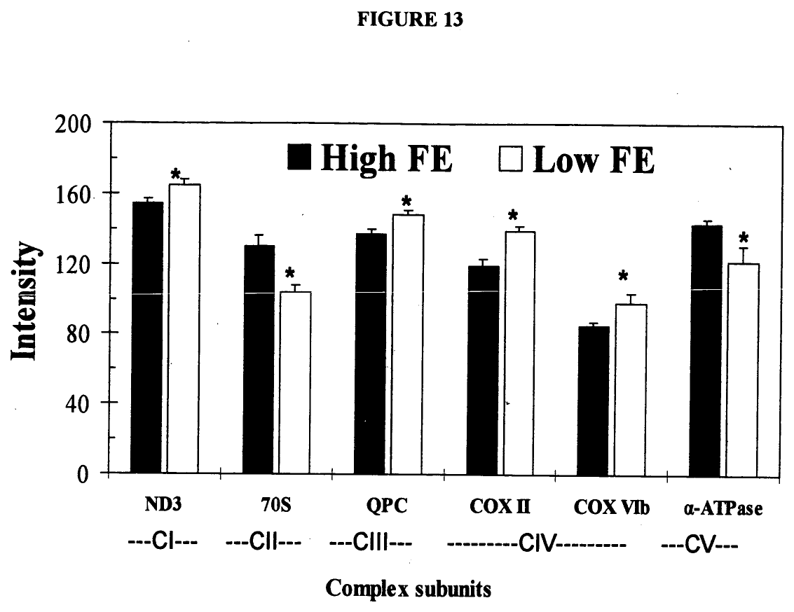

As the activities of the respiratory chain complexes could be related to expression of proteins within the individual complexes, several protein subunits in the respiratory chain complexes were detected using specific antibodies; six proteins were detected for Complex I [NAD3, NAD4, NAD5, NAD6 URF C, NAD6 URF L, NAD7 (mtDNA encoded subunits)], one protein for Complex II [70S (nDNA encoded subunit)], one for Complex IV [COX II (mtDNA encoded subunit)] and Complex V [α-ATPase (nDNA encoded subunits)], and five for Complex III [core 1, core IL cyt c1, ISP (nDNA encoded subunit) cyt b (mtDNA) encoded subunit. There were not differences in the expression of all the six protein subunits in Complex I, or in the expression of 70 S subunit of Complex II or the subunit of the Complex V between high and low FE mitochondria (Table 3). In contrast, higher expression of core L cyt c1, and cyt b were observed in low FE mitochondria but there were no differences in expression of ISP or core II between groups (see FIG. 13). The steady state level of COX II in the Complex IV was also higher (P=0.06) in low compared to high FE broilers.

TABLE 3
Mitochondrial Respiratory Chain Protein Subunits detected from Breast
Muscle in Broilers with Low and High Feed Efficiency (FE)¹
Mitochondrial Respiratory Chain
BAND INTENSITY

| Subunits (Complex)² | High FE (n = 7) | Low FE (n = 8) | P-value |
|---|---|---|---|
| NAD3 (I) | 98 ± 5 | 100 ± 4 | 0.90 |
| NAD4 (I) | 119 ± 4 | 118 ± 5 | 0.90 |
| NAD5 (I) | 145 ± 9 | 142 ± 11 | 0.90 |
| NAD6 URF C (I) | 118 ± 3 | 115 ± 6 | 0.68 |
| NAD6 URF C (I) | 158 ± 6 | 151 ± 9 | 0.50 |
| NAD6 URF C (I) | 89 ± 3 | 88 ± 3 | 0.10 |
| 70 S (FP) (II) | 105 ± 9 | 99 ± 11 | 0.70 |
| α-ATPASE (V) | 122 ± 8 | 116 ± 9 | 0.61 |

¹Mean ± SE of values shown in parentheses
²NAD ± mitochondrial adenine dinucleotide subunits 3 to 7; URF = unidentified reading frame; 70S = 70 subunit in complex II; FP = flavoprotein

All references cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### REFERENCES

1. Anderson, S., A. T. Bankier, B. G. Barrell, M. H. L. de Bruijn, A. R. Coulson, J. Drouin, I. C. Eperon, D. P. Nierlich. B. A. Roe, F. Sanger, P. H. Schreier, A. J. H. Smith, R. Staden, and I. G. Young, 1981. Sequence and organization of the human mitochondrial genome. Nature 290:457-465.
2. Baldwin, K. M., G. H. Klinkerfuss, R. L. Teijung, P. A. Mold, and J. O. Holloszy, 1972. Respiratory capacity of white, red, and intermediate muscle: adaptive response to exercise. Am. J. Physiol. 222: 373-378.
3. Bhattacharya, S. K., J. H. Thakar, J. H.; P. L. Johnson, and D. R. Shanklin. 1991. Isolation of skeletal muscle mitochondria from hamsters using an ionic medium containing ethylenediaminetetraacetic acid and nagarse. Anal. Biochem. 192: 344-349.
4. Bottje, W.; Tang, Z.; Iqbal, M; Cawthon, D.; Okomoto, R.; Wing, T.; Cooper, M. 2002. Association of mitochondrial function with feed efficiency within a single genetic line of male broilers. Poult. Sci., in press.
5. Boveris, A., and B. Chance, 1973. The mitochondrial generation of hydrogen peroxide. Biochem. J. 134: 707-711.
6. Chance, B.; Schoener, B., Oshino, R.; Itshak, R.; Nakase, Y. Oxidation-reduction ratio studies of mitochondria in freeze-trapped samples NADH and flavoprotein fluorescence signals. J. Biol. Chem. 1979, 254, 4764-71.
7. Cawthon D., R McNew, K. W. Beers, and W. G. Bottje, 1999. Evidence of mitochondrial dysfunction in broilers with pulmonary hypertension syndrome (Ascites): Effect of t-butyl hydroperoxide on function, glutathione and related thiols. Poultry Sci 78:114-125.
7. Cawthon, D., K. Beers, and W. G. Bottje, 2001. Electron transport chain defect and inefficient respiration may both underlie pulmonary hypertension syndrome (PHS)-associated mitochondrial dysfunction in broilers. Poultry Sci. 80:474-484.
8. Chamberlain, F. W., 1943. Atlas of Avian Anatomy. Michigan State Agricultural Experiment Station Hallenbeck Printing Co., Lansing Mich.
9. Chance, B., H. Sies, and A Boveris, 1979. Hydroperoxide metabolism in mammalian organs. Physiol. Rev. 59:527-605.
10. Desjardins, P., and R. Morais. 1990. Sequence and gene organization of the chicken mitochondrial genome. A novel gene order in higher vertebrates. J. Mol. Biol. 212: 599-634.
11. Emmerson, D. A., 1997. Commercial approaches to genetic selection for growth and feed conversion in domestic poultry. Poultry Sci. 76:1121-1125.
12. Estabrook, 1967. Mitochondrial respiratory control and the polarographic measurement of ADP: O ratios. Method Enzymology 10:41-47.
13. Field, J., H. S. Belding, and A. W. Martin, 1937. Evaluation of wet and dry weight bases for expression of respiratory rate in vitro. Proc. Soc. Exp. Biol. Med. 40:565-568.
14. Fiegal, R. J. and B. L. Shapiro, 1979. Mitochondrial calcium uptake and oxygen consumption in cystic fibrosis. Nature 278:276-77.
15. Galente, Y. M., and Y. Hatefi, 1978. Resolution of Complex I and isolation of NADH dehydrogenase and an iron-sulfur protein. Meth. Enzymol. 53: 15-21.
16. Hagen, T. D. L. Yowe, J. C. Bartholomew, C. M. Wehr, K. L. Do, J.-Y. Park, and B. N. Ames, 1997. Mitochondrial decay in hepatocytes from old rats: membrane potential declines, heterogeneity and oxidants increase. Proc. Natl. Acad. Sci. USA 94:3064-3069.

17. Hansford, R G., B. A. Hogue, and V. Mildaziene, 1997. Dependence of hydrogen peroxide formation by rat heart mitochondria on substrate availability and donor age. J. Bioenerg. Biomem. 29: 89-95.
18. Hatefi, Y., and D. L. Stiggall, 1978. Preparation and properties of succinate: ubiquinone reductase (Complex II). Meth. Enzymol. 53: 21-27.
19. Havenstein, G. B., P. R. Ferket, S. E. Scheidler, and B. T. Larson, 1994. Growth, livability, and feed conversion if 1957 and 1991 broilers when fed ‘typical’ 1957 and 1991 broiler diets. Poultry Sci. 73:1785-1794.
20. Herrero A., and G. Barja, 1998. Hydrogen peroxide production of heart mitochondria and aging rate are slower in canaries and parakeets than in mice: sites of free radical generation and mechanisms involved. Mech. Aging Devel. 103:133-146.
21. Hoppeler, H., Ol. Hudlicka, and E. Uhlmann, 1987. Relationship between mitochondria and oxygen consumption in isolated cat mitochondria. J. Physiol. 385:661-675.
22. Iqbal, M., D. Cawthon, R. F. Wideman, Jr., and W. G. Bottje, 2001. Lung mitochondrial dysfunction in pulmonary hypertension syndrome. I. Site specific defects in electron transport chain. Poultry Sci. 80:485-495.
23. Jackman, M. R., and W. T. Willis, 1996. Characteristics of mitochondria isolated from type I and type Iib skeletal muscle. Am. J. Physiol. 270:C673-C678.
24. Katayama, H.; Nagasu, T.; Oda, Y. 2001. Improvement of in-gel digestion protocol for peptide mass fingerprinting by matrix-assisted laser desorption/ionization time-of-flight mass spectrometry. Rapid Commun Mass Spectrum 15:1416-21.
25. Kristal, B., B. Park, and B. P. Yu, 1994. Antioxidants reduce peroxyl-mediated inhibition of mitochondrial transcription. Free Rad. Biol. Med. 16:653-660.
26. Kristal, B., S. Koopmans, C. T. Jackson, Y. Ikeno, B. Par, and B. P. Yu, 1997. Oxidant-mediated repression of mitochondrial transcription in diabetic rats. Free Rad. Biol. Med. 22:813-822.
27. Keller, R-J., Halmes, N. C., hinson, J. A. and Pumford, N. R. 1993. Immunochemical detection of oxidized proteins. Chem. Res. Toxicolo. 6:430-433.
28. Kwong, L. K., and R. S. Sohal, 1998. Substrate and site specificity of hydrogen peroxide generation in mouse mitochondria. Arch. Biochem. Biophys. 350:118-126.
29. Laemmli, U. K. 1970. Cleavage of structure proteins during assembly of the head of bacteriophage T4. Nature 227:680:685.
30. Lass, A., B. H. Sohal, R. Weindruch, M. J. Forster, and R. S. Sohal, 1998. Caloric restriction prevents age-associated accrual of oxidative damage to mouse skeletal muscle mitochondria. Free Rad. Biol. Med 25: 1089-1097.
31. Lehninger, A. L., D. L. Nelson, and M. M. Cox, 1993. In: Principles of Biochemistry 2d Ed., Worth Publishers, New York, N.Y. p. 558.
32. Mitsumoto, A., Takeuchi, A., Okawa, K., Nakagawa, Y. 2002. A subset of newly synthesized polypeptides in mitochondria from human endothelial cells exposed to hydroperoxide stress. Free Radic Biol. Med. 32:22-37.
33. Nohl, H., L. Gille, K. Schonheit, Y. Liu, 1996. Conditions allowing redox-cycling of ubisemiquinone in mitochondria to establish a direct redox couple with molecular oxygen. Free Rad. Biol. Med. 20:207-213.
34. O'Farrell, P. H. 1975. High resolution two-dimensional electrophoresis of proteins. J. Biol. Chem. 250:4007-21.
35. Pumford, N. R., Hinson, J. A., Benson, R. W., Roberts, D. W. 1990. Immunoblot analysis of proteins containing 3-(cystein-S-yl)acetaminophen adducts in serum and subcellular liver fractions from acetaminophen-treated mice. Toxicol Appl. Pharmacol. 104:521-32.
36. Robin, E., and R. Wang, 1988. Mitochondrial DNA molecules and virtual number of mitochondria per cell in mammalian cells. J. Cell Phys. 136:507-513.
37. Sue, C. M., and E. A. Schon, 2000. Mitochondrial chain diseases and mutations in nuclear DNA: A promising start. Brain Pathol. 10:442-450.
38. Tang, Z., M. Iqbal, D. Cawthon, and W. Bottje, 2001. Heart and Breast Muscle Mitochondrial Dysfunction in Broilers with Pulmonary Hypertension Syndrome. SPSS meeting Atlanta
39. Turrens, J. F., and A. Boveris, 1980. Generation of superoxide anion by the NADH dehydrogenase of bovine heart mitochondria. Biochem. J. 191:421-427.
40. Youlanda, M. A. R., and M. C. Blancher, 1970. Respiration and oxidative phosphorylation by mitochondria of red and with skeletal muscle. Can. J. Biochem. 48:27-32.

What is claimed is:

1. A method for predicting feed efficiency in an animal comprising:
(a) calculating feed efficiency of said animal comprising measuring feed intake and weight gain of said animal;
(b) obtaining a biological sample from said animal;
(c) calculating mitochondrial function of said animal comprising measuring oxygen consumption and electron transport activity;
(d) calculating a correlation between feed efficiency and mitochondrial function comprising comparing measured levels of feed efficiency from step (a) to measured levels of mitochondrial function from step (c);
(e) predicting the likelihood of high or low feed efficiency in said animal,
whereby a positive or negative direct relationship between said feed efficiency and said mitochondrial function of said correlation of step (d) indicates feed efficiency.

2. The method of claim 1, wherein said biological sample is selected from the group consisting of blood and tissue.

3. The method of claim 1, wherein calculating said mitochondrial function comprises measuring the activities of electron transport chain Complex I, II or both.

4. The method of claim 1, wherein calculating said mitochondrial function comprises measuring the ratio of the activities of electron transport chain Complex I and II.

5. The method of claim 1, wherein said biological is obtained in utero.

6. The method of claim 1, wherein said biological is obtained in ovo

7. A method for predicting feed efficiency in an animal comprising:
(a) calculating feed efficiency of said animal comprising measuring feed intake and weight gain of said animal;
(b) obtaining a biological sample from said animal;
(c) calculating mitochondrial function of said animal comprising measuring oxygen consumption and electron transport activity;
(d) calculating a correlation between feed efficiency and mitochondrial function comprising comparing the measured level of feed efficiency from step (a) to measured levels of mitochondrial function from step (c);
(e) obtaining protein patterns of said biological sample;
(f) comparing said protein patterns with said correlation of step (d)
(g) predicting the likelihood of high or low feed efficiency in said animal,
whereby a positive or negative direct relationship between said feed efficiency and said mitochondrial function of said correlation of step (d) and said protein patterns indicates feed efficiency.

8. The method of claim 7, wherein said biological sample is selected from the group consisting of blood and tissue.

9. The method of claim 7, wherein calculating said mitochondrial function comprises measuring the activities of electron transport chain Complex I, II or both.

10. The method of claim 7, wherein calculating said mitochondrial function comprises measuring the ratio of the activities of electron transport chain Complex I and II.

11. The method of claim 7, wherein said biological is obtained in utero.

12. The method of claim 7, wherein said biological is obtained in ovo

13. A method for predicting feed efficiency in an animal comprising:
(a) obtaining a biological sample from said animal;
(b) calculating mitochondrial function of said animal comprising measuring oxygen consumption and electron transport activity;
(c) obtaining protein patterns of said biological sample;
(d) analyzing said protein patterns;
(e) calculating a correlation between said mitochondrial function and said protein pattern comprising comparing the measured level of mitochondrial function from step (a) to the analysis of said protein patterns from step (d);
(f) predicting the likelihood of high or low feed efficiency in said animal,
whereby a positive or negative direct relationship between said mitochondrial function of said correlation of step (d) indicates feed efficiency.

14. The method of claim 13, wherein said biological sample is selected from the group consisting of blood and tissue.

15. The method of claim 13, wherein calculating said mitochondrial function comprises measuring the activities of electron transport chain Complex I, II or both.

16. The method of claim 13, wherein calculating said mitochondrial function comprises measuring the ratio of the activities of electron transport chain Complex I and II.

17. The method of claim 13, wherein said biological is obtained in utero.

18. The method of claim 13, wherein said biological is obtained in ovo

19. A kit for determining feed efficiency in a sample, said kit comprising:
(a) solid phase containing on its surface a plurality of antibodies each at a know location on said solid phase, each antibody capable of hybridizing to a protein derived therefrom said protein known to be increased or decreased in response to feed efficiency; and
(b) indicator linked to said antibodies wherein said indicator produces a color when said protein binds said antibodies;
(c) means for quantitating binding of said protein to said antibody; and
wherein said color is proportional to the amount of protein associated with feed efficiency present in said sample.

20. The kit of claim 19, wherein said sample is blood.

21. The kit of claim 19, wherein said indicator is a signal.

22. A method for predicting feed efficiency in an animal comprising:
(a) calculating feed efficiency of said animal comprising measuring feed intake and weight gain of said animal;
(b) obtaining a biological sample from said animal;
(c) calculating mitochondrial function of said animal comprising measuring oxygen consumption and electron transport activity;
(d) calculating a correlation between feed efficiency and mitochondrial function comprising comparing the measured level of feed efficiency from step (a) to measured levels of mitochondrial function from step (c);
(e) obtaining genetic patterns of said biological sample;
(f) comparing said genetic patterns with said correlation of step (d)
(g) predicting the likelihood of high or low feed efficiency in said animal,
whereby a positive or negative direct relationship between said feed efficiency and said mitochondrial function of said correlation of step (d) and said genetic patterns indicates feed efficiency.

23. The method of claim 22, wherein said biological sample selected from the group consisting of RNA, DNA and nucleic acid fragments.

24. A kit for determining feed efficiency in a sample, said kit comprising:
(a) solid phase containing on its surface a plurality of nucleic acid of different sequences, each at a known location on said solid phase, each nucleic acid capable of hybridizing to an RNA species or cDNA derived therefrom, said RNA species known to be increased or decreased in response to feed efficiency;
(b) indicator linked to said nucleic acid wherein said indicator produces a color when said RNA or cDNA species binds said nucleic acid species;
(c) means for quantitating binding of said RNA or cDNA species to said nucleic,
wherein said color is proportional to the amount of gene associated with feed efficiency present in said sample.

25. The kit of claim 24, wherein said sample is blood.

26. The kit of claim 24, wherein said sample is tissue.

27. The kit of claim 24, wherein said indicator is a signal.
